(19) United States
(12) Patent Application Publication
(10) Pub. No.: US 2002/0198118 A1
(43) Pub. Date: Dec. 26, 2002

(54) Cream soap with paste-like consistency

(76) Inventor: Edmund D. George, Warwick, RI (US)

Correspondence Address: Dike, Bronstein, Roberts & Cushman, Intellectual Property Practice Group, Edwards & Angell, LLP, P.O. Box 9169, Boston, MA, 02209, US

(21) Appl. No.: 10/161,186

(22) Filed: Jun. 3, 2002

Related U.S. Application Data
(60) Provisional application No. 60/345,027, filed on Jan. 4, 2002.
(60) Provisional application No. 60/297,303, filed on Jun. 11, 2001.

(51) Int. Cl. A61K 7/50

(52) U.S. Cl. 510/130; 510/152; 510/155; 510/158; 510/481

(57) ABSTRACT

An improved cream soap is disclosed. The cream soap is comprised of water, a surfactant/soap system, one or more detergents, a preservative, and at least one structuring/emulsifying agent. Generally, the cream soap composition further comprises one or more of each of a colorant, a fragrance, and a skin benefit additive. The soap has a more paste-like consistency as compared to conventional cream soaps. And by virtue of its paste-like consistency, this soap can be stored in, and dispensed from a wider array of containment units, and also avoids the problem of “run off” that plagues conventional cream soaps.

### CROSS REFERENCE TO RELATED APPLICATIONS

This application claims priority from, and incorporates by reference the entirety of U.S. Provisional Patent Application Serial No. 60/297,303, which was filed on Jun. 11, 2001, and U.S. Provisional Patent Application Serial No. 60/345,027, which was filed on Jan. 4, 2002.

### BACKGROUND OF THE INVENTION

1. Field of the Invention

The present invention relates generally to cleansing products and, more particularly, to cream soaps with a paste-like consistency that exhibits excellent cleaning prowess and that may be stored in, and dispensed from, a wider variety of containers as compared to conventional cream soaps.

2. Background

Few can debate the utility of washing one's hands with soap, a practice which medical experts have long recognized as being one of, if not the most simple yet effective methods of ridding one's hands of dirt, germs and bacteria.

Most individuals aspire to wash their hands regularly, but find that doing so often has adverse consequences that, in their opinion, outweigh the utility of frequent hand washing. Among the reasons most often cited as explanations for why people are dissuaded from hand washing are that the process renders their hands dry and itchy to the touch, as well as red in appearance, and that the soap leaves an unpleasant odor on their hands.

A complaint unique to bar-type soaps is that after only a few uses, bar soaps often become water-logged, which, in turn, renders them tactilely unpleasant. Also, visually unattractive surface cracks and blotching can develop on bar soaps as they dry following use.

An alternative to conventional bar soaps are glycerine soaps, which are bar soaps cast from a molten liquid soap. Glycerine soaps, however, suffer from some, if not all, of the disadvantages of conventional bar soaps and, in addition, tend to have a high dissolving rate. This, in turn, causes glycerine soap bars to be “used-up” very quickly, thus rendering such soaps cost inefficient.

Among the alternatives to bar soaps are cream-based soaps. Generally, the majority of such cream soaps are placed into plastic containers from which they are dispensed on a use-by-use basis to hand washers, who then simply wet the soap with water to wash their hands. Only a small amount of the cream soap is dispensed to a user, with the remainder being housed in the container. This use-by-use dispensing system is advantageous because it allows cream soaps to avoid the aforementioned problems associated with bar-type soaps.

Unfortunately, cream soaps suffer from several disadvantages, mostly owing to their relatively low viscosity. For example, cream soaps often have a low enough viscosity that they are prone to “run-off,” wherein the soap drips off a user's hands prior to, or during, the hand washing process. This run off, in turn, can contact clothes and/or leave a surface residue in the vicinity of hand washing environments, both of which are undesirable. Also, cream soap that “runs off” is unused, which is uneconomical.

Another problem directly attributable to the relatively low viscosity of cream soaps is that it is not feasible and/or practical to store such soaps in a number of desirable storage/dispensing containers.

For example, conventional cream soaps generally are not stored in tubes or bottles because of likelihood of encountering the “run off” problem upon attempting to access the soap. Moreover, conventional cream soaps also tend not to be stored in other desirable dispensing containers (e.g., jars) due to realistic fear of spillage prior to or while users “scoop out” the soap from the container.

Therefore, a need exists for a cream soap that exhibits excellent cleansing prowess, but that, as compared to known cream soaps, can be dispensed from a wider array of containers.

### SUMMARY OF THE INVENTION

These, and other needs, are met by the present invention, which provides a cream soap comprised of water, a surfactant/soap system, one or more detergents, a preservative, and at least one structuring/emulsifying agent. Generally, but not necessarily, the cream soap composition further comprises one or more of each of a colorant, a fragrance, and a skin benefit additive.

By including such ingredients in predetermined weight percentages, a cream soap in accordance with the present invention exhibits superb cleansing prowess, excellent foaming and pay-off characteristics, and, following its use, leaves the skin clean, yet also with a soft, smooth and healthy feel and appearance.

Such a cream soap also has a more paste-like consistency as compared to conventional cream soaps. And by virtue of its paste-like consistency, this soap avoids the problem of “run off” that plagues conventional cream soaps. Moreover, its comparatively paste-like form versus conventional cream soaps also enables this soap to be housed in, and dispensed from a wider array of containment units.

In an exemplary aspect of the present invention, the bulk of the composition of the cream soap comprises water. To allow the cream soap to attain an ideal paste-like consistency, water should comprise in the range of about 32.2% to 63% by weight of the overall composition of the cream soap.

A cream soap in accordance with the present invention also should contain at least one detergent, which provides cleansing prowess to the soap. Preferably, more than one detergent is included in the cream soap in order to bestow the properties of each detergent on the soap. However, regardless of the type and number of detergents present in the cream soap, it/they should comprise, in total, in the range of about 16% to 28% by weight of the overall composition of the cream soap.

Another ingredient in the cream soap is a surfactant/soap system, which provides additional cleansing prowess to the soap. Currently, it is preferred for the surfactant/soap system of the cream soap to be comprised of a blend of two sodium salts of triglycerides. A blend of these sodium salts not only provides the soap with excellent cleansing prowess, but also with an increased rate of foam/lather generation (due to the inclusion of sodium cocoate powder), and a more enduring foam/lather (due to the inclusion of sodium palmate).

Regardless of the type and number of surfactant/soap(s) that actually comprise the system, it/they should comprise, in total, in the range of about 8% to 14% by weight of the overall composition.

A cream soap of the present invention also should include at least one emulsifying/structuring agent in order to prevent/deter any potential physical separation of the soap's water-based and primarily oil-based ingredients. In an exemplary aspect of the present invention, a cream soap includes, as emulsifying/structuring agents, three distinct acid-based agents.

Generally, the weight percentage of each emulsifying/structuring agent is in the range of about 2% to 11% of the overall composition of the cream soap.

In addition to these ingredients, a cream soap of the present invention also includes one or more preservatives, which serve to protect against decomposition, spoilage and/or rancidity of the soap's ingredients and, therefore, of the soap itself. Also, it is beneficial to include a preservative due the soap containing a relatively high percentage of water, which is a medium for bacteriological growth.

Generally, however, only a small amount (e.g., about 0.5%) of the soap's composition should comprise a preservative. This is because inclusion of a higher weight percentage of preservatives would not provide additional protection against decomposition, but would necessitate reducing the amount(s) of other ingredients.

Optionally, yet preferably, a cream soap in accordance with the present invention includes one or more additives that benefit the skin. Regardless of the type and number of skin benefiting additives present in the cream soap, it/they should comprise, in total, in the range of about 0% to 4% by weight of the overall composition of the cream soap.

Optionally, but again preferably, a cream soap in accordance with the present invention further includes one or more fragrances or perfumes that provide the soap with a pleasant odor during and after use. When present, such fragrance(s)/perfume(s) account(s) for up to about 2% by weight of the overall composition of the soap.

A cream soap in accordance with the present invention may also be treated, as is generally known in the art, with one or more colorants to provide the cream soap with a desired color.

### DETAILED DESCRIPTION OF THE INVENTION

In an exemplary embodiment of the present invention, a cream soap is comprised of water, a surfactant/soap system, one or more detergents, a preservative, and at least one structuring/emulsifying agent. Generally, the cream soap composition further comprises one or more of each of a colorant, a fragrance, and a skin benefit additive.

Table I illustrates some of the potential ingredients of a cream soap according to the present invention, and approximate weight percentage options/ranges (with respect to the overall composition of the soap) for each ingredient.

By including these ingredients in these amounts, a cream soap in accordance with the present invention exhibits superb cleansing prowess, excellent foaming and pay-off characteristics, and, following its use, leaves the skin clean, yet also with a soft, smooth and healthy feel and appearance.

Such a soap also has a more paste-like consistency as compared to conventional cream soaps. And by virtue of its paste-like consistency, this soap avoids the problem of “run off” that plagues conventional cream soaps. Moreover, its comparatively paste-like form versus conventional cream soaps also enables this soap to be housed in, and dispensed from a wider array of containment units.

TABLE I

| | | | Currently |
|---|---|---|---|
| | Range of | Range of | Most |
| | Acceptable | Preferred | Preferred |
| | Weight | Weight | Weight |
| Ingredient | Percentages | Percentages | Percentage |
| Water | 32.3%-63% | 37.5%-58.5% | 47.75% |
| Soap System | 8%-14% | 8%-12% | 10% |
| Detergent | 16%-28% | 18.5%-24% | 21.75% |
| Structuring | 12.5%-22% | 14%-20.5% | 17.25% |
| and/or | | | |
| Emulsifying | | | |
| Agent(s) | | | |
| Preservative(s) | 0.5% | 0.5% | 0.5% |
| Skin Benefit | 0%-4% | 1%-3% | 2% |
| Additive(s) | | | |
| Fragrance(s) | 0%-2% | 0.5%-1.5% | 0.75% |

As indicated in this Table, the bulk of the composition of the cream soap comprises water. Accordingly, variation of the weight percentage of water in the soap is the most significant determinate of the soap's viscosity and, therefore, whether or not it assumes its desired paste-like consistency.

For example, if the soap is comprised of too much water, it will have a low viscosity (i.e., it will not be paste-like), and, therefore, will be subject to undesirable “run off,” and will be incapable of storage within, and/or being dispensed from certain containers (e.g., jars, bottles and tubes). If, instead, the soap contains too little water, it will be extremely thick (i.e., too paste-like) and, therefore, will not be amenable to storage within, and/or being dispensed from certain other containers (e.g., pumps and mousses).

To allow the soap to attain an ideal paste-like consistency, water should comprise in the range of about 32.2% to 63% by weight of the overall composition of the cream soap, with a range of about 37.5% to 58.5% by weight being preferred, and a weight percentage of about 47.75% being currently most preferred.

A cream soap in accordance with the present invention also should contain at least one detergent, which provides cleansing prowess to the soap. Ideally, the detergent should also be mild enough to avoid causing/promoting skin dryness and/or redness following use of the soap.

Among the classes of suitable mild, yet cleansing detergents are surfactants, such as amphoteric, anionic and nonionic surfactants, with anionic surfactants being preferred detergent ingredients for a cream soap of the present invention. Suitable anionic surfactants to add to the cream soap include, but are not limited to, sodium cocoyl isethionate and sodium coco glyceryl sulfonate.

Preferably, more than one detergent is included in the cream soap in order to bestow the properties of each detergent on the soap. It is currently preferred to include two detergents, e.g., sodium cocoyl isethionate and sodium coco glyceryl sulfonate, in the cream soap.

Regardless of the type and number of detergents present in the cream soap, it/they should comprise, in total, in the range of about 16% to 28% by weight of the overall composition of the cream soap, with a range of about 18.5% to 24% by weight being preferred, and a weight percentage of about 21.75% being currently most preferred.

Another ingredient in the cream soap is a surfactant/soap system, which provides additional cleansing prowess to the soap.

Numerous surfactants and soaps can be included as part of the surfactant/soap system. Among the suitable surfactants/soaps are sodium or potassium salts (or a blend of sodium and potassium salts) of triglycerides and/or fatty acids. Exemplary trigylcerides and fatty acids include, but are not limited to, palm oil, coconut oil, palm kernel oil, and beef tallow.

Currently, it is preferred for the surfactant/soap system of the cream soap to be comprised of a blend of two sodium salts of triglycerides. Two currently preferred sodium salts are sodium palmate (i.e., a sodium salt of palm oil) and sodium cocoate powder (i.e., a sodium salt of coconut oil). A blend of these sodium salts not only provides the soap with excellent cleansing prowess, but also with an increased rate of foam/lather generation (due to the inclusion of sodium cocoate powder), and a more enduring foam/lather (due to the inclusion of sodium palmate).

Regardless of the type and number of surfactant/soap(s) that actually comprise the system, it/they should comprise, in total, in the range of about 8% to 14% by weight of the overall composition of the cream soap, with a range of about 8% to 12% by weight being preferred, and a weight percentage of about 10% currently being most preferred.

A cream soap of the present invention also should include at least one emulsifying/structuring agent in order to prevent/deter any potential physical separation of the soap's water-based and primarily oil-based ingredients.

In other words, the presence of the emulsifying/structuring agent(s) should ensure that the soap has a uniform physical consistency, despite the soap being comprised of many ingredients, some of which have different polarities, which, but for the presence of the emulsifying/structuring agent(s), would not blend together to form a soap of generally uniform texture and consistency.

Numerous types of emulsifying/structuring agents may be included in the cream soap, with fatty acid-based agents and fatty alcohols, being some examples of such agents.

Suitable acid-based agents include, but are not limited to, acid glycol esters (such as C18-36 acid glycol esters), acid triglycerides (such as C18-36 acid triglyderides), and fatty acids (such as stearic acid), while an exemplary fatty alcohol is cetyl alcohol.

In an exemplary embodiment of the present invention, a cream soap includes, as emulsifying/structuring agents, three distinct acid-based agents, wherein these preferred acid-based agents are C18-36 acid glycol ester, stearic acid, and C18-36 acid triglycerides.

In such an embodiment, the weight percentages of the C18-36 acid glycol esters, the stearic acid, and the C18-36 acid triglycerides comprise, respectively, in the range of about 2.25% to 5% (preferably about 2.5% to 4.5%, currently most preferably about 3%), about 3.75% to 6.55% (preferably about 4% to 6%, currently most preferably about 5.25%), and about 6.5% to 10.75% (preferably about 7.5% to 10%, currently most preferably about 9%) of the overall composition of the cream soap.

In addition to these ingredients, a cream soap of the present invention also includes one or more preservatives, which serve to protect against decomposition, spoilage and/or rancidity of the soap's ingredients and, therefore, of the soap itself. Also, it is beneficial to include a preservative due the soap containing a relatively high percentage of water, which is a medium for bacteriological growth.

As indicated in the Table above, only a small amount (about 0.5%) of the soap's composition should comprise a preservative. This is because inclusion of a higher weight percentage of preservatives would not provide additional protection against decomposition, but would necessitate reducing the amount(s) of other ingredients.

Exemplary preservatives may be natural (e.g., benzoic acid, carrot oil, Vitamin E oil, and grapefruit seed extract) and/or synthetic (e.g., sodium hydroxymethylglycinate, butylparaben, methylparaben), with synthetic preservatives being currently more preferred.

Optionally, yet preferably, a cream soap in accordance with the present invention includes one or more additives that benefit the skin. Such additives may include, but are not limited to, sea kelp extracts (which act as skin soothers), clays (which absorb oil from the skin), fresh water silt (which soothes and remineralizes the skin), alpha-bisabolol (an anti-inflammatory ingredient), vitamins, exfoliants, anti-microbial agents, and microencapsulations.

Regardless of the type and number of skin benefiting additives present in the cream soap, it/they should comprise, in total, in the range of about 0% to 4% by weight of the overall composition of the cream soap, with a range of about 1% to 3% by weight being preferred, and a weight percentage of about 2% being currently most preferred.

Optionally, but again preferably, a cream soap in accordance with the present invention further includes one or more fragrances or perfumes that provide the soap with a pleasant odor during and after use. When present, such fragrance(s)/perfume(s) account(s) for up to about 2% (preferably in the range of about 0.5% to 1.5 percent, currently most preferably about 0.75%) by weight of the overall composition of the soap.

Examples of suitable fragrances include, but are not limited to, floral, citrus, fruit, botanical, and chamomile fragrances, and essential oils such as patchouli, peppermint, chamomile, and eucalyptus.

A cream soap in accordance with the present invention may also be treated, as is generally known in the art, with one or more colorants to provide the cream soap with a desired color. Exemplary colorants are dyes, and/or pigments, and/or colors that are either certified by the Food and Drug Administration (FDA), non-certified by the FDA, or not subject to certification by the FDA.

In an embodiment in which one or more colorants comprise part of the overall composition of the cream soap, the weight percentages indicated in the Table above are adjusted (i.e., reallocated) to allow for the inclusion this ingredient, and/or for the inclusion of additional additives, including, but not limited, to those listed above. This may be accomplished by renormalizing the total composition of the cream soap. Alternatively, the weight percentage of soap/surfactant system can be reduced pro rata to account for the additional weight percentage of this/these other ingredient(s). As noted in the Table above, however, the weight percentage of the surfactant/soap system should not be less than 64% of the overall composition of the cream soap, so as not to compromise the cream soap's cleansing prowess.

Due to the presence of the above-indicated amounts of these ingredients, a cream soap in accordance with the present invention exhibits superb cleansing prowess, excellent foaming and pay-off characteristics, and, following its use, leaves the skin with a clean, yet soft, smooth and healthy feel and appearance.

Additionally, a cream soap having such a composition is somewhat paste-like in form due to its high viscosity. And due to its paste-like consistency, this soap avoids the problem of “run off” that plagues conventional cream soaps, and may be stored in, and dispensed from a wider array of containment units.

For example, whereas prior art cream soaps are almost uniformly housed in pump-actuated containers, cream soaps in accordance with the present invention can be housed in such containers, but also in mousses, tubes, jars, and bottles, the latter two containment units being particularly undesirable storage media for conventional cream soaps.

In addition to the ingredients noted above for the cream soap, other exemplary ingredients (in lieu of, and/or in addition to, any of those listed above) may include those listed/referenced in the Cosmetic, Toiletry, and Fragrance Association (CTFA) International Cosmetic Ingredient Dictionary and Handbook, McCutcheon's Volume 1 (Emulsifiers & Detergents), McCutcheon's Volume 2 (Functional Materials), and/or other comparable sources.

The foregoing description of the invention is merely illustrative thereof, and it is understood that variations and modifications can be effected without departing from the scope or spirit of the invention as set forth in the following claims. All publications referenced herein are incorporated by reference in their entirety.

What is claimed is:

1. A cream soap having an overall composition, the cream soap comprising:
a predetermined quantity of water that comprises in the range of about 32.2% to 63% by weight of the overall composition of the cream soap;
at least one detergent;
a surfactant system;
at least one emulsifying agent; and
at least one preservative.

2. The cream soap of claim 1, wherein the at least one detergent comprises, in total, at least about 16% by weight of the overall composition of the cream soap.

3. The cream soap of claim 1, wherein the surfactant system comprises at least about 8% by weight of the overall composition of the cream soap.

4. The cream soap of claim 1, wherein the at least one emulsifying agent comprises, in total, at least about 12.5% by weight of the overall composition of the cream soap.

5. The cream soap of claim 1, wherein at least one preservative comprises, in total, about 0.5% by weight of the overall composition of the cream soap.

6. The cream soap of claim 1, further comprising:
at least one additional additive, wherein each additive is selected from the group consisting of skin benefiting ingredients, fragrances, and colorants.

7. The cream soap of claim 1, wherein the surfactant system is a sodium salt of a triglyceride.

8. The cream soap of claim 1, wherein the surfactant system is a sodium salt of a fatty acid.

9. The cream soap of claim 1, wherein the surfactant system is comprised of a blend of sodium palmate and sodium cocoate powder.

10. The cream soap of claim 1, wherein each of the at least one emulsifying agents is selected from the group consisting of fatty acid-based emulsifying agents and fatty alcohol emulsifying agents.

11. The cream soap of claim 1, wherein the cream soap includes a plurality of emulsifying agents, at least one of which is fatty acid-based.

12. The cream soap of claim 1, wherein each of the at least one preservative is a synthetic preservative.

13. The cream soap of claim 1, wherein the predetermined quantity of water comprises in the range of about 37.5% to 58.5% by weight of the overall composition of the cream soap.

14. A cream soap having an overall composition, the cream soap comprising:
a predetermined quantity of water that comprises in the range of about 37.5% to 58.5% by weight of the overall composition of the cream soap;
at least one detergent;
a surfactant system;
a plurality of emulsifying agents; and
at least one preservative.

15. The cream soap of claim 14, further comprising:
at least one additional additive, wherein each additive is selected from the group consisting of skin benefiting ingredients, fragrances, and colorants.

16. The cream soap of claim 14, wherein at least one of the plurality of emulsifying agents is a fatty acid-based emulsifying agent.

17. The cream soap of claim 14, wherein each of at least one preservative is a synthetic preservative.

18. The cream soap of claim 14, wherein the surfactant system is a sodium salt of a triglyceride.

19. The cream soap of claim 14, wherein the surfactant system is a sodium salt of a fatty acid.

20. A cream soap having an overall composition, the cream soap comprising:
a predetermined quantity of water that comprises in the range of about 32% to 63% by weight of the overall composition of the cream soap;
a plurality of detergents;
a surfactant system;
a plurality of emulsifying agents;
a preservative; and
at least one additional additive, wherein each additional additive is selected from the group consisting of skin benefiting ingredients, fragrances, and colorants.
